(19) United States
(12) Patent Application Publication
(10) Pub. No.: US 2025/0381314 A1
(43) Pub. Date: Dec. 18, 2025

(54) METHODS FOR DISINFECTING CONTACT LENSES USING A COMBINATION OF MANGANESE AND IRON CATALYSTS

(71) Applicant: Better Vision Solutions LLC, Alpharetta, GA (US)

(72) Inventor: Lynn Winterton, Alpharetta, GA (US)

(21) Appl. No.: 19/237,201

(22) Filed: Jun. 13, 2025

Related U.S. Application Data
(60) Provisional application No. 63/659,385, filed on Jun. 13, 2024.

(51) Int. Cl. A61L 12/12; A61L 12/08; A61L 101/28; A61L 101/34; A61L 101/48

(52) U.S. Cl. CPC A61L 12/128; A61L 12/088; A61L 2101/28; A61L 2101/34; A61L 2101/48

(57) ABSTRACT

Contact lens treatment systems and methods for disinfecting contact lenses are described. These systems and methods utilize a coated catalytic disc in combination with a fibrous catalyst. The coated catalytic disc contains a support disc and 6 to 300 mg of manganese present on at least a portion of the support disc, often in the form of manganese dioxide. The fibrous catalyst contains 25 to 50 mg iron, often in the form of an iron cation.

### REFERENCE TO RELATED APPLICATION

This application claims the benefit of U.S. Provisional Application No. 63/659,385, filed on Jun. 13, 2024, the disclosure of which is incorporated herein by reference in its entirety.

### FIELD OF THE INVENTION

The present disclosure concerns methods for disinfecting contact lenses and contact lens treatment systems, and more particularly relates to the use of a manganese-coated catalytic disc in combination with an iron fibrous catalyst for such methods and systems.

### BACKGROUND OF THE INVENTION

Contact lens treatment systems of various styles and functionality are utilized for storing, cleaning and/or disinfecting contact lenses. In some lens containment and care systems, a hydrogen peroxide (H₂O₂) based disinfecting solution is used, in which the contact lenses to be cleaned and/or disinfected are deposited in contact with the hydrogen peroxide disinfecting solution for a certain period of time, such that cleaning and/or disinfecting of the contact lenses can take place to a satisfactory extent. The contact lens treatment systems utilized for cleaning and/or disinfecting of contact lenses typically contain a lens basket, which opens to receive the contact lenses to be cleaned and closes to retain the lenses during treatment. The lens basket can be integrated as part of the cap, which can be attached to a suitable container. Before the cap is placed onto the container, the container is dosed with a desired amount of the hydrogen peroxide disinfecting solution. The contact lens basket containing the lenses to be treated is immersed into the hydrogen peroxide disinfecting solution in the container, and the container is closed by securing the cap onto the container.

Conventionally, a platinum disc is present in the container, often secured to the cap and lens basket, to decompose the hydrogen peroxide. It would be beneficial to develop alternative and improved contact lens treatment systems for disinfecting contact lenses that do not contain platinum. Accordingly, it is to this end that the present invention is generally directed.

### SUMMARY OF THE INVENTION

This summary is provided to introduce a selection of concepts in a simplified form that are further described below in the detailed description. This summary is not intended to identify required or essential features of the claimed subject matter. Nor is this summary intended to be used to limit the scope of the claimed subject matter.

One aspect of this invention is directed to methods for disinfecting a contact lens. For instance, a typical method for disinfecting a contact lens can comprise immersing the contact lens in a volume of from 2 mL to 20 mL of a disinfecting solution comprising from 1.5 vol % to 5 vol % H₂O₂ in a container in the presence of (i) a coated catalytic disc and (ii) a fibrous catalyst comprising iron. In certain aspects, the coated catalytic disc can comprise a polymer support disc and a manganese oxide on at least a portion of the support disc. The catalytic disc can comprise from 6 mg to 300 mg of manganese. The fibrous catalyst can be present in an amount from 0.1 g to 0.35 g and contain from 25 mg to 50 mg of iron per gram of the fibrous catalyst.

Contact lens disinfecting systems also are disclosed herein. A representative contact lens disinfecting system can comprise (a) a container configured to receive a cleaning solution, (b) a cap removably attachable to the container, (c) any of the coated catalytic discs comprising any amount of manganese disclosed herein, and (d) any of the fibrous catalysts comprising any amount of iron disclosed herein.

Both the foregoing summary and the following detailed description provide examples and are explanatory only. Accordingly, the foregoing summary and the following detailed description should not be considered to be restrictive. Further, features or variations may be provided in addition to those set forth herein. For example, certain aspects may be directed to various feature combinations and sub-combinations described in the detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific aspects presented herein.

FIG. 1 illustrates a top and front perspective view of a contact lens disinfecting system consistent with an aspect of this invention.

FIG. 2 illustrates a side perspective view of an integrated cap, lens basket, and coated catalytic disc shown in FIG. 1.

FIG. 3 illustrates a perspective assembly view of a contact lens disinfecting system consistent with another aspect of this invention.

FIG. 4 illustrates a front perspective view of an integrated cap, lens basket, and coated catalytic disc shown in FIG. 3.

FIGS. 5A-5B are an exploded view and a bottom exploded view, respectively, of the coated catalytic disc shown in FIG. 4.

FIG. 6 presents hydrogen peroxide decomposition plots for the manganese oxide coated catalytic discs of Example 1.

### DEFINITIONS

To define more clearly the terms used herein, the following definitions are provided. Unless otherwise indicated, the following definitions are applicable to this disclosure. If a term is used in this disclosure but is not specifically defined herein, the definition from the IUPAC Compendium of Chemical Terminology, 2^{nd} Ed (1997), can be applied, as long as that definition does not conflict with any other disclosure or definition applied herein, or render indefinite or non-enabled any claim to which that definition is applied. To the extent that any definition or usage provided by any document incorporated herein by reference conflicts with the definition or usage provided herein, the definition or usage provided herein controls.

Herein, features of the subject matter are described such that, within particular aspects, a combination of different features can be envisioned. For each and every aspect and/or feature disclosed herein, all combinations that do not detrimentally affect the products, compositions, systems, and methods described herein are contemplated with or without explicit description of the particular combination. Additionally, unless explicitly recited otherwise, any aspect and/or feature disclosed herein can be combined to describe inventive features consistent with the present disclosure.

In this disclosure, while products, compositions, systems, and methods are often described in terms of “comprising” various components, materials, devices, or steps, the products, compositions, systems, and methods also can “consist essentially of” or “consist of” the various components, materials, devices, or steps, unless stated otherwise.

The terms “a,” “an,” and “the” are intended to include plural alternatives, e.g., at least one. For instance, the disclosure of “a contact lens” is meant to encompass one or more than one contact lens (e.g., two contact lenses), unless otherwise specified. Herein, “contact lens” is used generically to encompass any ophthalmic lens.

Several types of ranges are disclosed in the present invention. When a range of any type is disclosed or claimed, the intent is to disclose or claim individually each possible number that such a range could reasonably encompass, including end points of the range as well as any sub-ranges and combinations of sub-ranges encompassed therein. For example, when the present disclosure recites that the coated catalytic disc can contain from 0.1 wt. % to 2 wt. % manganese (Mn), the intent is to disclose or claim individually every possible number that such a range could encompass, consistent with the disclosure herein. For example, the disclosure that the coated catalytic disc contains from 0.1 wt. % to 2 wt. % manganese (Mn), as used herein, discloses that the manganese content can be any amount within the range and, for example, can be equal to 0.1, 0.2, 0.4, 0.6, 0.8, 1, 1.2, 1.4, 1.6, 1.8, or 2 wt. % Mn, as well as any range between these two numbers (for example, the amount of Mn can be in a range from 0.5 wt. % to 1.5 wt. %), and also including any combination of ranges between these two numbers (for example, 0.1 to 0.5 wt. % and 0.7 to 1.3 wt. %).

Although any methods, devices, and materials similar or equivalent to those described herein can be used in the practice or testing of the invention, the typical methods, devices, and materials are herein described.

All publications and patents mentioned herein are incorporated herein by reference in their entirety or the purpose of describing and disclosing, for example, the constructs and methodologies that are described in the publications and patents, which might be used in connection with the presently described invention.

### DETAILED DESCRIPTION OF THE INVENTION

Disclosed herein are peroxide-based contact lens treatment systems and methods for disinfecting contact lenses. Unexpectedly, it was found that a combination of a manganese oxide coated disc and a fibrous catalyst comprising iron can result in an improvement over conventional methods relying on expensive platinum catalyst technology.

### Coated Catalytic Discs

The coated catalytic disc consistent with aspects of this invention can comprise a support disc, and a manganese oxide present on at least a portion of the support disc. Generally, a uniform coating of the manganese oxide is applied to the support disc, such that the manganese oxide is present on a large portion of the support disc, and in some instances, substantially all of the support disc is coated with the manganese oxide. Thus, the manganese oxide can be present on at least 50%, at least 85%, at least 90%, at least 95%, at least 98%, or at least 99% of the surface area of the support disc.

The coated catalytic disc typically contains from 6 mg to 300 mg of manganese. This amount is based on elemental manganese, not the manganese oxide. In one aspect, the coated catalytic disc can contain from 20 mg to 280 mg of manganese, while in another aspect, the coated catalytic disc can contain from 60 mg to 250 mg of manganese, from 100 mg to 250 mg of manganese in yet another aspect, and from 150 mg to 225 mg of manganese in still another aspect. Other appropriate ranges for the amount of manganese present on the coated catalytic disc are readily apparent from this disclosure.

While not limited thereto, the coated catalytic disc can contain from 0.1 wt. % to 2 wt. % manganese. The weight percentage is determined by dividing the total weight of manganese (elemental) on the coated catalytic disc by the weight of the support disc. In some aspects, the coated catalytic disc can contain from 0.2 wt. % to 2 wt. % manganese; alternatively, from 0.2 wt. % to 1.8 wt. % manganese; alternatively, from 0.5 wt. % to 1.5 wt. % manganese; or alternatively, from 0.7 wt. % to 1.3 wt. % manganese. Other appropriate ranges for the percentage amount of manganese present on the coated catalytic disc are readily apparent from this disclosure.

The volume, surface area, and weight of the coated catalytic disc (or the polymer support disc) are not particularly limited. Generally, however, the volume of the coated catalytic disc (or the support disc) falls within a range from 0.5 cc to 1 cc, from 0.6 cc to 0.9 cc, or from 0.6 cc to 0.8 cc. As one of skill in the art would readily recognize, the volume can depend upon the geometry (shape and size) of the disc. Likewise, the surface area of the coated catalytic disc (or the support disc) is not limited to any particular range, but often falls within a range from 2 cm² to 40 cm², from 10 cm² to 30 cm², or from 15 cm² to 25 cm². As evidenced by the low surface area, the support disc has no internal porosity, or substantially no internal porosity. In addition to disc geometry, the density of the support disc can impact the weight of the coated catalytic disc (or the support disc).

Illustrative and non-limiting ranges for the weight of the coated catalytic disc (or the support disc) include from 0.2 g to 4 g, from 0.3 g to 2 g, from 0.5 g to 1.5 g, or from 0.5 g to 1 g. Additionally, or alternatively, the coated catalytic disc (or the support disc) can have a specific surface area (in units of cm²/g) in a range from 1 cm²/g to 20 cm²/g, such as, for instance, from 2 cm²/g to 18 cm²/g, from 5 cm²/g to 15 cm²/g, or from 7 cm²/g to 12 cm²/g. Other appropriate ranges for the volume, surface area, weight, and specific surface area of the coated catalytic disc (or the support disc) are readily apparent from this disclosure.

The coated catalytic disc can comprise a manganese oxide deposited on at least a portion of the support disc. Any suitable manganese oxide can be used, non-limiting examples of which can include manganese (II) oxide (MnO), manganese (III) oxide (Mn₂O₃), manganese dioxide (MnO₂), and the like, as well as any combination thereof. In particular aspects of this invention, the manganese oxide can comprise (or consist essentially of, or consist of) manganese dioxide (MnO₂).

The polymer support disc can be constructed of any material suitable for supporting manganese. Generally, the support disc is prepared from (often injection molded from) a polymer. Thus, while not being limited thereto, the support disc can comprise a polyamide (PA), a polyphenylene oxide (PPO), a polyphenyl ether (PPE), a polypropylene (PP), a polyethylene (PE), a polystyrene (PS), and the like, as well as any mixture or combination thereof. A particular suitable polymer type for the support disc is a NORYL resin, although not limited thereto.

### Methods for Making the Coated Catalytic Disc

Aspects of this invention are directed to methods of making the coated catalytic disc, for example, the coated catalytic disc as described hereinabove. Such methods can comprise contacting a polymer support disc with a coating composition (e.g., a paste) comprising a binder, a manganese oxide (e.g., in flake form), and a diluent; and drying and/or curing to form the coated catalytic disc. Generally, the features of the methods disclosed herein (e.g., the support disc, the binder, the manganese oxide, the diluent, the conditions under which the support disc and the coating composition are contacted, and the drying or curing conditions, among others) are independently described herein, and these features can be combined in any combination to further describe the disclosed methods. Moreover, other process steps can be conducted before, during, and/or after any of the steps listed in the disclosed methods, unless stated otherwise. Coated catalytic discs prepared by any of these methods also are encompassed herein.

The coating composition can contain a binder, a manganese oxide (e.g., manganese dioxide in flake form), and a diluent. Any suitable binder can be used, such as an acrylic polymer, a urethane polymer, or any combination thereof. Likewise, any suitable diluent can be used, such as water, an organic solvent (e.g., mineral spirits, turpentine, an alcohol, etc.), or any combination thereof. In some aspects, the coating composition is a “water-based” coating composition, and the diluent comprises (or consists essentially of, or consists of) water. Generally, the organic solvent is not particularly limited, so long as it does not adversely impact the support disc (e.g., solubilize) or the binder.

While not limited thereto, the coating composition can contain from 0.05 wt. % to 5 wt. % of the manganese oxide. The weight percentage is determined by dividing the weight of the manganese oxide by the total weight of the coating composition. In some aspects, the coating composition can contain from 0.3 wt. % to 3 wt. % manganese oxide; alternatively, from 0.5 wt. % to 2 wt. % manganese oxide; or alternatively, from 0.1 wt. % to 1 wt. % manganese oxide. Other appropriate ranges for the percentage amount of manganese oxide present in the coating composition are readily apparent from this disclosure.

Optionally, the coating composition can contain any suitable inert filler, non-limiting examples of which can include carbon black, calcium carbonate, titanium dioxide, a pigment, and the like, as well as combinations thereof. When present, the coating composition can contain from 0.1 wt. % to 10 wt. % of the inert filler. The weight percentage is determined by dividing the weight of the inert filler by the total weight of the coating composition. In some aspects, the coating composition can contain from 0.3 wt. % to 8 wt. % filler; alternatively, from 0.5 wt. % to 4 wt. % filler; or alternatively, from 1 wt. % to 3 wt. % filler. Other appropriate ranges for the percentage amount of inert filler present in the coating composition are readily apparent from this disclosure.

In the first step of the process, the support disc can be contacted with the coating composition, which can often be in the form of a mixture or slurry of solids in the diluent (e.g., a paste). Methods of contacting can include dipping the disc in, immersing the disc in, enrobing the disc with, tumbling the disc with, blending the disc with, spraying the disc with, brushing the disc with, or coating the disc with, the coating composition, for example, to form a coating or layer on at least a portion (or all or substantially all) of the support disc. Combinations of more than one method of contacting the disc and the coating composition can be used.

In one aspect, the support disc (or discs) can be enrobed with the coating composition by placing the disc (or discs) under a waterfall of the coating composition to apply a layer of the coating composition on at least a portion (or all or substantially all) of the support disc. Excess coating composition can drip off the disc due to gravity or be removed by any other suitable technique.

In another aspect, the disc (or discs) can be placed in a mesh basket, which is rotated through a vessel containing the slurry coating composition, thereby dipping or immersing the disc (or discs) in the coating composition. One rotation through the coating composition can be used, but often several rotations are used. Excess coating can drip off the disc due to gravity or be removed by any suitable technique.

After contacting the support disc with the coating composition, drying (or curing) is used to form the coated catalytic disc. The wet/coated discs can be dried (or cured) in a basket or on a rack, although not limited to. Illustrative drying (or curing) conditions can include a temperature from 20° C. to 150° C. or from 25° C. to 80° C., at ambient pressure or any suitable sub-atmospheric pressure. Thus, in one aspect, drying (or curing) can be conducted at ambient temperature and pressure. Higher temperatures can be used, but generally not so high as to adversely impact or decompose the binder or the support disc.

### Fibrous Catalysts

For the avoidance of doubt, by the term “fibrous catalyst” we mean a catalyst that comprises polymer fibers to which catalytically active sites or centers are attached. By the term “fibers” we include both a single monofilament and a complex filament that is made up of more than one monofilament. An example fibrous catalyst can be any fabric that includes polyacrylonitrile (hereinafter “PAN”) fibers. The references herein to a fabric may refer simply to an arrangement of one or more PAN fibers. In one aspect of the invention, the fabric that comprises PAN fibers is a knitted fabric, such as a fibrous knitted mesh. Thus, in this aspect, the PAN fibers/yarn are capable of being knitted. The knitted fabric may be prepared by any suitable method known in the art.

The example fibrous catalyst can be prepared using a process including the steps of (i) treating a fabric comprising polyacrylonitrile fibers with a hydrazine salt selected from hydrazine sulfate and dihydrazine sulfate and hydroxylamine sulfate in the presence of a base to provide a modified fabric, (ii) treating the modified fabric with a base, and (iii) treating the modified fabric with an aqueous solution comprising a sulfate salt of an iron cation and a salt (e.g., a sulfate, nitrate and/or chloride salt) of a second metal cation, wherein the second metal cation is selected from a lithium, sodium, potassium, magnesium, calcium and zinc cation, and mixtures thereof. Example fibrous catalysts used in this method can include fibrous catalysts described in U.S. Pat. Nos. 8,410,011 and 8,513,303, and U.S. Patent Publication Number 2011/0098174.

The fibrous catalyst can comprise an iron cation. In certain aspects, the iron cation can be selected from Fe²⁺ and Fe³⁺, or a combination thereof. The iron may be impregnated within the fibrous catalyst, or alternatively, may be covalently bound to the fibrous catalyst. As disclosed herein, the amount of iron within the fibrous catalyst can exceed 20 mg of iron per gram of fabric, and in certain aspects can be in a range from 25 mg to 50 mg, 25 mg to 40 mg, or 25 mg to 35 mg of iron (or iron cation) per gram of the fibrous catalyst.

### Methods for Disinfecting Contact Lenses

Aspects of this invention are directed to methods for disinfecting a contact lens, and such methods can comprise immersing the contact lens in a volume of a disinfecting solution comprising from 1.5 vol % to 5 vol % H₂O₂ in the presence of (i) a coated catalytic disc, for example, the coated catalytic disc as described hereinabove, and (ii) a fibrous catalyst comprising iron, also as described above.

Generally, the features of the methods disclosed herein (e.g., the concentration of H₂O₂ in the disinfecting solution, the volume of the disinfecting solution, the coated catalytic disc, and the fibrous catalyst, among others) are independently described herein, and these features can be combined in any combination to further describe the disclosed methods. Moreover, other process steps can be conducted before, during, and/or after any of the steps listed in the disclosed methods, unless stated otherwise. For instance, and optionally, an enzyme tablet can be added to the disinfecting solution before or during immersing the contact lens.

The disinfecting solution generally is configured to disinfect the contact lens. Typically, the disinfecting solution contains from 1.5 vol % to 5 vol % H₂O₂, but in some aspects of this invention, the disinfecting solution can contain from 3 vol % to 4 vol % or from 2.5 vol % to 3.5 vol %, H₂O₂. The volume of the disinfecting solution used to treat the contact lens (or lenses) is not particularly limited, and the appropriate volume of the disinfecting solution can be defined by the container (having a removable cap and lens basket) in which the disinfection process is conducted. Nonetheless, representative and non-limiting volumes of the disinfecting solution can range from 2 mL to 20 mL in one aspect, from 2 mL to 15 mL in another aspect, from 5 mL to 15 mL in yet another aspect, and from 8 mL to 12 mL in still another aspect.

The coated catalytic disc used in the methods for disinfecting a contact lens can be any coated catalytic disc disclosed herein, for example, comprising a support disc and a manganese oxide present on at least a portion of the support disc, in which the coated catalytic disc contains from 6 mg to 300 mg of manganese. The coated catalytic disc can be configured to neutralize (or decompose) the disinfecting solution (e.g., convert most of the hydrogen peroxide to water and oxygen), typically within a time period that suits the contact lens wearer.

In certain aspects, between 0.01 g and 5 g of the fibrous catalyst is present in the disinfecting solution. In other aspects, the amount of fibrous catalyst present can be in a range from 0.05 g to 0.1 g, from 0.1 g to 0.5 g, or from 0.25 g to 0.5 g. The relative amounts of fibrous catalyst disclosed herein can be taken in conjunction with the amounts of catalytic discs and the respective amounts of manganese present to form a suitable ratio between the manganese and iron present in the disinfecting solution for the disinfection methods disclosed herein. In certain aspects, the molar ratio of Mn:Fe can be from 0.01:1 to 150:1, from 1:1 to 100:1, from 3:1 to 70:1, or from 10:1 to 50:1.

In certain aspects, the fibrous catalyst can comprise an amount of Fe of greater than 1 mg per gram of fabric, greater than 10 mg per gram of fabric, greater than 18 mg per gram of fabric, or greater than 25 mg per gram of fabric; alternatively in a range from 18 mg to 100 mg per gram of fabric. Thus, in certain aspects, an amount of Fe employed within the disinfecting solution can be in a range from 1 mg to 500 mg, from 10 mg to 250 mg, or from 25 mg to 100 mg.

The contact lens (or lenses) can be immersed for a time period of at least 2 hr, at least 4 hr, from 1 hr to 12 hr, or from 2 hr to 6 hr, although other time intervals can be used. In some aspects, the contact lens (or lenses) can be immersed for a time period sufficient to reduce the H₂O₂ concentration of the disinfecting solution to less than 100 ppm, and in some instances, less than 50 ppm, or less than 25 ppm (ppm by volume). The disinfecting solution, after neutralization, often can have a pH in a range from 6 to 8, or from 6.5 to 7.5, but is not limited thereto.

### Contact Lens Disinfecting Systems

Various contact lens disinfecting systems are disclosed and described herein. For instance, the contact lens disinfecting system can comprise (a) a container configured to receive a disinfecting solution, (b) a cap removably attachable to the container, (c) a coated catalytic disc, and (d) a fibrous catalyst, including for example, the coated catalytic disc and fibrous catalyst as described hereinabove. Generally, the features of the contact lens disinfecting system disclosed herein (e.g., the container, the cap, the coated catalytic disc, and the fibrous catalyst, among others) are independently described herein, and these features can be combined in any combination to further describe the disclosed contact lens disinfecting systems. Moreover, other parts or components can be present in the disclosed contact lens disinfecting systems, unless stated otherwise.

The coated catalytic disc (any coated catalytic disc disclosed herein) can be attached to the cap to form a unitary structure or an integrated structure. In some aspects, the disc can be removably attached to the cap, while in other aspects, the disc can be fixedly attached to the cap. Further, the cap can comprise a lens basket for retaining a contact lens (or lenses), and the lens basket can be configured to project into the container. In particular aspects of this invention, the lens basket is positioned between the coated catalytic disc and the top of the cap. Additionally, or alternatively, the fibrous catalyst may be positioned at the bottom of the container.

The container and the cap can be constructed of any suitable material (typically, a polymer or plastic material), and have any suitable geometry. For instance, the container can be generally cylindrical in shape. In some aspects of this invention, the cap can be configured to snap onto the container, while in other aspects, the cap can be configured to screw on and off the container. As to the latter option, the container can have an opening with an external threaded portion, and the cap can have an internal threaded portion. If desired, the cap can additionally contain a pressure-relief vent.

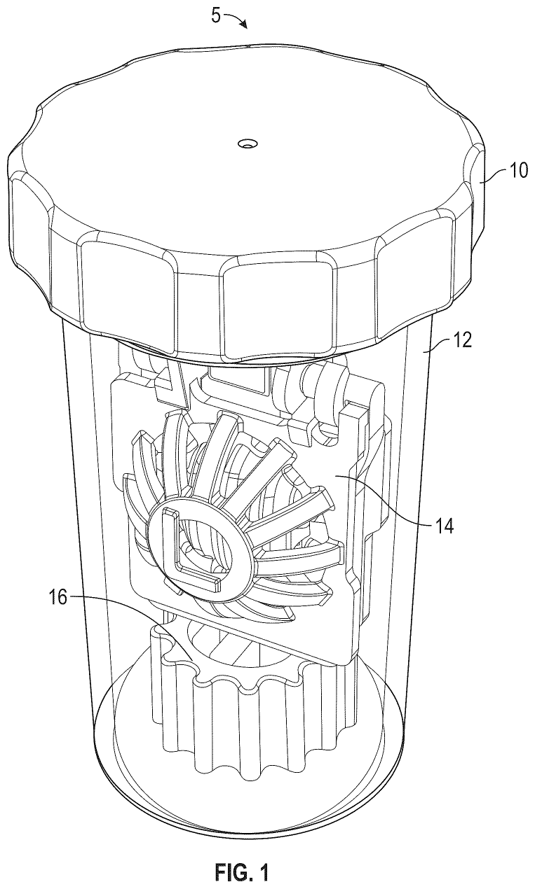

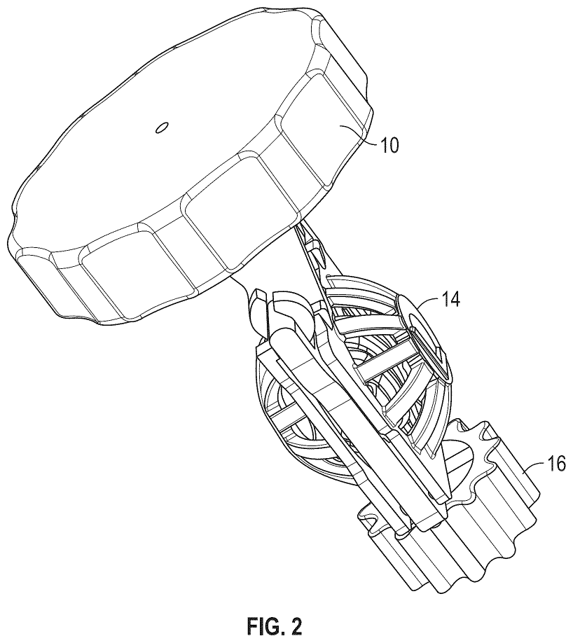

A representative contact lens disinfecting system 5 consistent with aspects of this invention is illustrated in FIG. 1. The contact lens disinfecting system 5 includes a cap 10 that connects with a container 12, preferably to form a seal. A lens basket 14 extends from and is supported by the cap 10. In use, the lens basket 14 receives contact lenses and is inserted into the container 12. A coated catalytic disc 16 is connected to the distal free end of the cap 10. FIG. 2 illustrates a unitary construction of the cap 10 with integrated lens basket 14 and coated catalytic disc 16.

In use, the container 12 is filled with a disinfecting solution containing hydrogen peroxide, as described herein. The lens basket 14 is inserted into the disinfecting solution in the container 12 such that the contact lenses are immersed. The hydrogen peroxide disinfects the contact lenses, and the coated catalytic disc 16 neutralizes the hydrogen peroxide.

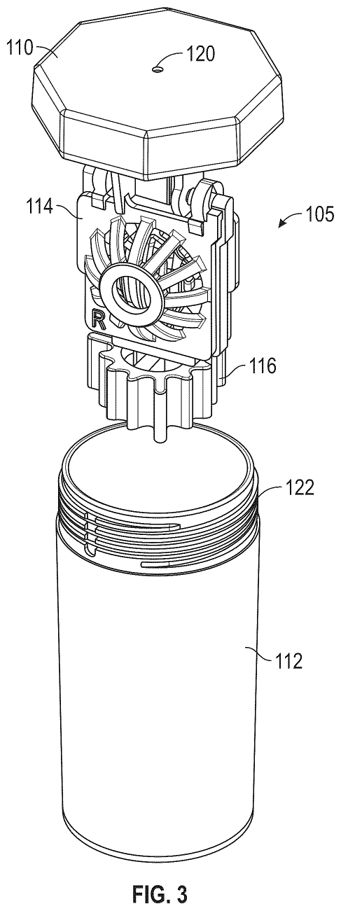

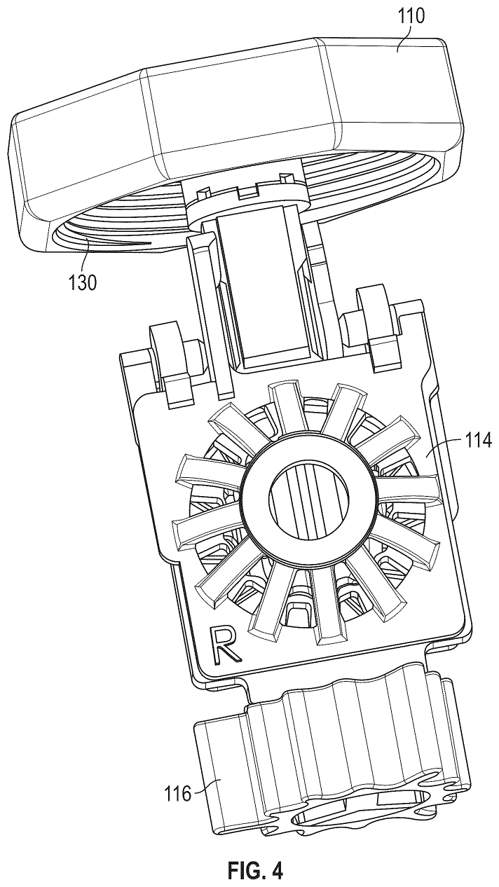

Another representative contact lens disinfecting system 105 consistent with aspects of this invention is illustrated in FIG. 3, and includes a cap 110, a container 112, a lens basket 114, and a coated catalytic disc 116. FIG. 4 illustrates a unitary construction of the cap 110 with integrated lens basket 114 and coated catalytic disc 116. The cap 110 can have a pressure-relief vent 120 and an internal threaded portion 130, and the container 112 can have an external threaded portion 122.

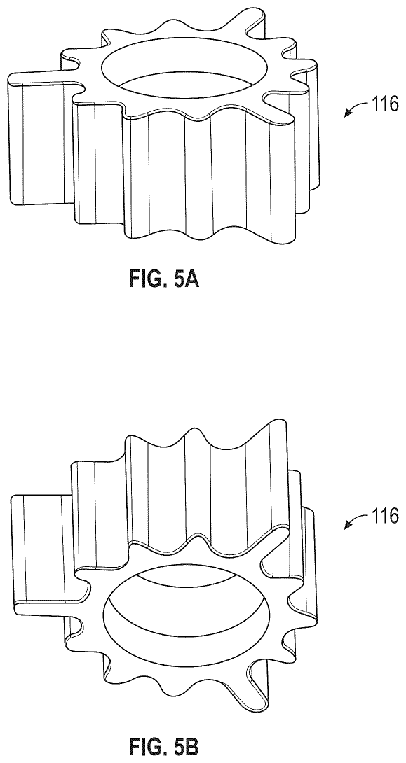

FIGS. 5A-5B show expanded views of the coated catalytic disc 116, and the different geometry as compared to the coated catalytic disc in FIG. 2. Any suitable disc geometry (shape or size) can be used in the contact lens disinfecting system, such as described in U.S. Patent Publication No. 2011/0114517. The use of the coated catalytic disc (containing manganese) is not limited solely to the disinfecting systems disclosed herein and can be integrated into any conventional contact lens disinfecting system, such as described in U.S. Pat. Nos. 6,945,389, 8,767,367, and 9,532,632.

### EXAMPLES

The invention is further illustrated by the following example, which is not to be construed in any way as imposing limitations to the scope of this invention. Various other aspects, modifications, and equivalents thereof which, after reading the description herein, can suggest themselves to one of ordinary skill in the art without departing from the spirit of the present invention or the scope of the appended claims.

### Example 1

For Example 1, the following coating formulation was used to prepare manganese coated catalytic discs from injection molded NORYL polymer support discs: 21 g of a 1 wt. % aqueously-solubilized high molecular weight acrylic polymer, 49 g deionized (DI) water, 55 g manganese dioxide (CAS 1313-13-9), and 5 g gamma alumina (γ-AlO₂).

At room temperature and pressure, approximately one-half of the water was placed into a glass beaker. All of the acrylic polymer solution was added to the DI water and stirred with a magnetic stirrer for 10-15 min. The remainder of the DI water was then added and stirred for 10-15 min. All of the manganese dioxide was then added and stirred for 5-10 min. This coating formulation was allowed to stand for 5-10 min, and the consistency was a thick, yet flowing (not thixotropic), mixture. The γ-AlO₂ was then added all at one time. Rapid mixing with a paddle was begun since the coating formulation became thick very quickly. After stirring for 1-12 min, the coating formulation was allowed to stand for 3-5 min. This coating formulation had the texture of a thixotropic paste.

Then, 100 NORYL support discs were added to a lapidary (rock) tumbler. For this volume, an MJR six-pound tumbler was sufficient (MJR Tumblers, 4316 N Gocke Road, Pecatonica, IL 61063). The 100 NORYL support discs supports were added to the empty six-pound tumbling barrel. Using a lab-spatula, all of the coating formulation was added to the tumbling barrel. The barrel was capped and placed on the mechanical roller. The roller was started and allowed to tumble the support discs and coating formulation for 1 hr.

A sheet of parchment baking-type liner was placed onto a wire cooling rack. The tumbler was opened and all of the coated catalytic discs were poured onto the lined cooling rack. Using forceps, the discs were separated from each other upon the cooling rack. This cooling rack was then placed into a preheated 80° C. oven for 2 hr for curing. The rack was then removed from the oven and allowed to cool to room temperature before using any of the coated catalytic discs.

A coated catalytic disc, as shown in FIGS. 5A-5B, was connected to the distal free end of the cap assembly as shown in FIG. 4. Ten mL of ClearCare™ hydrogen peroxide solution (3 vol %) was added to the container (see FIG. 3) and the cap assembly (with lens basket and coated catalytic disc, see FIG. 3) was lowered and placed into the peroxide solution. The cap was screwed closed in a finger-tight manner and left to stand for 6 hr. During this time, evolution of oxygen gas was observed as the hydrogen peroxide was neutralized to water and oxygen.

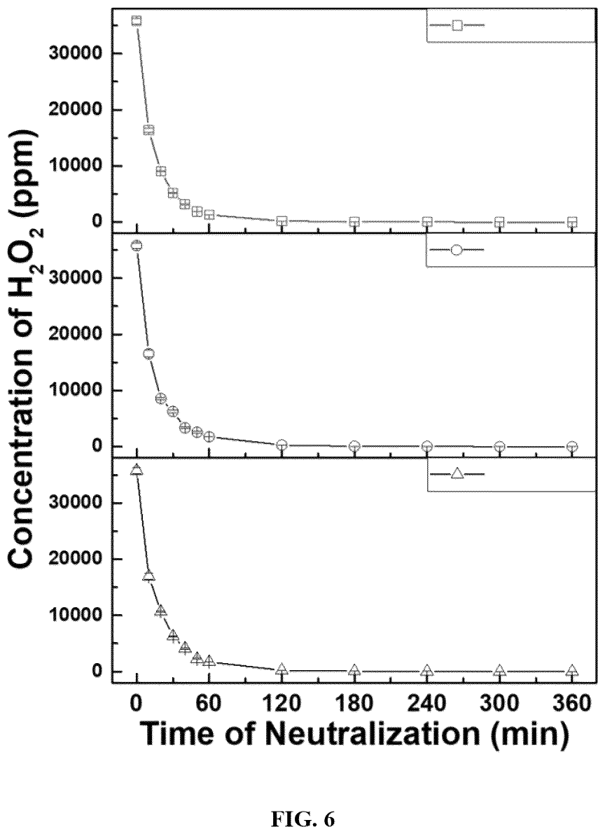

FIG. 6 illustrates the peroxide decomposition results of three replicates. Unexpectedly, the manganese coated catalytic discs of Example 1 had excellent peroxide decomposition kinetics. After 6 hr of neutralization time, the residual H₂O₂ concentration was less than 50 ppm.

### Example 2

For Example 2, manganese coated catalytic discs were prepared as described for Example 1 above.

The coated catalytic disc, as shown in FIGS. 5A-5B, was connected to the distal free end of the cap assembly as shown in FIG. 4. A pad of catalytic fabric impregnated with iron was prepared as described from U.S. Pat. No. 8,410,011 B2. Generally, a fibrous knitted mesh consisting of polyacrylonitrile (PAN) complex fibres and polypropylene (PP) inert mono-fibres was knitted as reported in GB-A-2,346,569, then modified in a three-stage process. At the first treatment stage, 30 g of fabric was immersed into an autoclave containing 800 ml of an aqueous solution of 30 g/l hydrazine dihydrochloride, 42 g/l hydroxylamine monohydrochloride and sodium hydroxide in the amount required for a pH of 9.5. The fabric was maintained at a temperature of from 101 to 102° C. for 2 hours, then it was taken out and washed with distilled water.

At the second treatment stage, the fabric was treated for 30 seconds with boiling aqueous NaOH solution having a concentration of 50 g/l followed by subsequent washing with distilled water. The ratio of the solution mass and knitted mesh mass was equal to 800 ml/30 g. At the third treatment stage, the fabric (30 g) was immersed into 1050 ml of an aqueous metal salt solution of FeCl₃·6H₂O for 19 hours. The catalyst was then ready to use after washing with distilled water and drying at room temperature.

The catalytic fabric prepared as above and catalytic fabric (0.5 g fabric, containing 20 mg Fe) was placed on the bottom of the container, along with 10 ml of ClearCare™ hydrogen peroxide solution (3 vol %) (see FIG. 3). The cap assembly (with lens basket and coated catalytic disc, see FIG. 3) was lowered and placed into the peroxide solution. The cap was screwed closed in a finger-tight manner and left to stand for 6 hr. During this time, evolution of oxygen gas was observed as the hydrogen peroxide was neutralized to water and oxygen. After 6 hr, the residual concentration of H₂O₂ within the peroxide solution was measured. The peroxide was then replaced and the experiment repeated for a total of 30 cycles.

The results of each neutralization cycle are shown in Table I below. As shown, each cycle was neutralized over the period of 6 hr, from an initial concentration in a range from 3.0 wt. % to 3.6 wt. % to an amount less than 60 ppm. In aggregate, cycles began with a starting concentration of 3.31 wt. %+/−0.17 wt. % and were reduced to a residual concentration of 45.76 ppm+/−5.98 ppm.

TABLE I

| | Baseline | Residual |
|---|---|---|
| | H₂O₂ conc. | H₂O₂ conc. |
| Cycle | (wt. %) | (ppm) |
| Cycle 1 | 3.22 | 37.00 |
| Cycle 2 | 3.14 | 43.00 |
| Cycle 3 | 3.07 | 46.67 |
| Cycle 4 | 3.06 | 44.33 |
| Cycle 5 | 3.07 | 37.33 |
| Cycle 6 | 3.14 | 43.33 |
| Cycle 7 | 3.13 | 42.67 |
| Cycle 8 | 3.40 | 32.00 |
| Cycle 9 | 3.20 | 42.33 |
| Cycle 10 | 3.42 | 36.00 |
| Cycle 11 | 3.42 | 53.00 |
| Cycle 12 | 3.09 | 39.33 |
| Cycle 13 | 3.60 | 55.33 |
| Cycle 14 | 3.47 | 57.00 |
| Cycle 15 | 3.12 | 34.00 |
| Cycle 16 | 3.02 | 55.00 |
| Cycle 17 | 3.54 | 59.33 |
| Cycle 18 | 3.42 | 55.00 |
| Cycle 19 | 3.55 | 49.67 |
| Cycle 20 | 3.40 | 44.33 |
| Cycle 21 | 3.57 | 39.67 |
| Cycle 22 | 3.42 | 39.67 |
| Cycle 23 | 3.37 | 34.67 |
| Cycle 24 | 3.42 | 47.00 |
| Cycle 25 | 3.49 | 45.00 |
| Cycle 26 | 3.46 | 42.33 |
| Cycle 27 | 3.40 | 45.67 |
| Cycle 28 | 3.18 | 58.67 |
| Cycle 29 | 3.22 | 56.67 |
| Cycle 30 | 3.28 | 56.67 |

### Example 3

A solution of the neutralized hydrogen peroxide solution was prepared according to the procedure of Example 2. Broadly, a Lens Case was charged with 10 mL of a 3 wt. % H₂O₂ solution containing the lens holder comprising the catalytic disc (containing Mn) and the catalytic fabric (containing Fe) as described above. After 6 hours, the neutralized solution was confirmed not to induce ocular irritability in rabbits. The neutralized solution also was determined to have no cytotoxic potential to L929 cells.

### Aspects and Embodiments

The invention is described above with reference to numerous aspects and specific examples. Many variations will suggest themselves to those skilled in the art in light of the above detailed description. All such obvious variations are within the full intended scope of the appended claims. Other aspects of the invention can include, but are not limited to, the following (aspects are described as “comprising” but, alternatively, can “consist essentially of” or “consist of” unless specifically stated otherwise):
Aspect 1. A method for disinfecting a contact lens, the method comprising:
immersing the contact lens in a volume of from 2 mL to 20 mL of a disinfecting solution comprising from 1.5 vol % to 5 vol % H₂O₂ in a container in the presence of:
(i) a coated catalytic disc comprising:
a polymer support disc; and
a manganese oxide on at least a portion of the support disc, wherein the coated catalytic disc comprises from 6 mg to 300 mg of manganese; and
(ii) from 0.1 g to 0.35 g of a fibrous catalyst comprising from 25 mg to 50 mg of iron per gram of the fibrous catalyst.
Aspect 2. The method defined in aspect 1, wherein the amount of manganese is from 60 mg to 250 mg, from 100 mg to 250 mg, or from 150 mg to 225 mg.
Aspect 3. The method defined in any aspect 1 or 2, wherein the manganese oxide comprises manganese (II) oxide (MnO), manganese (III) oxide (Mn₂O₃), manganese dioxide (MnO₂), or any combination thereof; preferably MnO₂.
Aspect 4. The method defined in any one of aspects 1-3, wherein the fibrous catalyst is positioned at a bottom of the container.
Aspect 5. The method defined in any one of aspects 1-4, wherein the fibrous catalyst comprises polyacrylonitrile (PAN) fibers.
Aspect 6. The method defined in any one of aspects 1-5, wherein the amount of the fibrous catalyst is from 0.1 g to 0.25 g or from 0.15 g to 0.25 g.
Aspect 7. The method defined in any one of aspects 1-6, wherein the fibrous catalyst comprises from 25 mg to 40 mg or from 25 mg to 35 mg of iron per gram of the fibrous catalyst.
Aspect 8. The method defined in any one of aspects 1-7, wherein the iron is covalently bound to the fibrous catalyst.
Aspect 9. The method defined in any one of aspects 1-8, wherein the iron is an iron cation (Fe²⁺ and/or Fe³⁺).
Aspect 10. The method defined in any one of aspects 1-9, wherein the molar ratio of Mn:Fe is from 0.01:1 to 150:1, from 1:1 to 100:1, from 3:1 to 70:1, or from 10:1 to 50:1.
Aspect 11. The method defined in any one of aspects 1-10, wherein the contact lens is immersed for at least 2 hr, from 1 hr to 12 hr, or from 2 hr to 6 hr.
Aspect 12. The method defined in any one of aspects 1-11, wherein the contact lens is immersed for a time sufficient to reduce the H₂O₂ concentration to less than 100 ppm, less than 50 ppm, or less than 25 ppm.
Aspect 13. The method defined in any one of aspects 1-12, wherein the disinfecting solution, after neutralization, has a pH in a range from 6 to 8, or from 6.5 to 7.5.
Aspect 14. The method defined in any one of aspects 1-13, wherein the volume of the disinfecting solution is from 2 mL to 15 mL, from 5 mL to 15 mL, or from 8 mL to 12 mL.
Aspect 15. The method defined in any one of aspects 1-14, wherein the coated catalytic disc is configured to neutralize the disinfecting solution.
Aspect 16. The method defined in any one of aspects 1-15, wherein the disinfecting solution is configured to disinfect the contact lens.
Aspect 17. The method defined in any one of aspects 1-16, wherein the disinfecting solution comprises from 3 vol % to 4 vol % or from 2.5 vol % to 3.5 vol. % H₂O₂.
Aspect 18. The method defined in any one of aspects 1-17, wherein the polymer support disc comprises a polyamide (PA), a polyphenylene oxide (PPO), a polyphenyl ether (PPE), a polypropylene (PP), a polyethylene (PE), a polystyrene (PS), or any combination thereof.
Aspect 19. The method defined in any one of aspects 1-18, wherein the coated catalytic disc (or the polymer support disc) has a volume in a range from 0.5 cc to 1 cc or from 0.6 cc to 0.9 cc.
Aspect 20. The method defined in any one of aspects 1-19, wherein the coated catalytic disc (or the polymer support disc) has a surface area in a range from 2 cm² to 40 cm², from 10 cm² to 30 cm², or from 15 cm² to 25 cm².
Aspect 21. The method defined in any one of aspects 1-20, wherein the coated catalytic disc (or the polymer support disc) weighs from 0.2 g to 4 g, from 0.5 g to 1.5 g, or from 0.5 g to 1 g.
Aspect 22. The method defined in any one of aspects 1-21, wherein the coated catalytic disc (or the polymer support disc) has a specific surface area in a range from 1 cm²/g to 20 cm²/g or from 5 cm²/g to 15 cm²/g.
Aspect 23. The method defined in any one of aspects 1-22, wherein the coated catalytic disc contains from 0.1 wt. % to 2 wt. % Mn, from 0.5 wt. % to 1.5 wt. % Mn, or from 0.7 wt. % to 1.3 wt. % Mn.
Aspect 24. The method defined in any one of aspects 1-23, wherein the coated catalytic disc is prepared by a process comprising (a) contacting the polymer support disc with a coating composition (paste) comprising a binder, a manganese oxide in flake form, and a diluent; and (b) drying/curing to form the coated catalytic disc.
Aspect 25. The method defined in aspect 24, wherein the binder comprises an acrylic polymer and/or a urethane polymer.
Aspect 26. The method defined in aspect 24 or 25, wherein the diluent comprises water.
Aspect 27. The method defined in any one of aspects 24-26, wherein contacting comprises dipping, immersing, enrobing, tumbling, blending, spraying, brushing, coating, or any combination thereof (to form a coating on the polymer support disc).
Aspect 28. The method defined in any one of aspects 24-27, wherein drying/curing comprises a temperature from 20° C. to 150° C. or from 25° C. to 80° C. at ambient pressure or any suitable sub-atmospheric pressure.
Aspect 29. The method defined in any one of aspects 24-28, wherein the coating composition comprises from 0.05 wt. % to 5 wt. % or from 0.1 wt. % to 1 wt. % of the manganese oxide.
Aspect 30. The method defined in any one of aspects 24-29, wherein the coating composition further comprises an inert filler.
Aspect 31. A contact lens disinfecting system comprising:
(a) a container configured to receive a disinfecting solution;
(b) a cap removably attachable to the container;
(c) a coated catalytic disc as defined in any one of aspects 1-3 or 18-30; and
(d) a fibrous catalyst as defined in any one of aspects 1 or 4-10.
Aspect 32. The system defined in aspect 31, wherein the coated catalytic disc is removably attached to the cap.
Aspect 33. The system defined in aspect 31 or 32, wherein the fibrous catalyst is positioned at a bottom of the container.
Aspect 34. The system defined in any one of aspects 31-33, wherein the cap further comprises a lens basket for retaining a contact lens, and wherein the lens basket is configured to project into the container.
Aspect 35. The system defined in any one of aspects 31-34, wherein the cap comprises a pressure-relief vent.
Aspect 36. The system defined in any one of aspects 31-35, wherein the container comprises an opening with an external threaded portion.
Aspect 37. The system defined in any one of aspects 31-36, wherein the cap comprises an internal threaded portion.
Aspect 38. The system defined in any one of aspects 31-37, wherein the cap is configured to screw on and off the container.
Aspect 39. The system defined in any one of aspects 31-35, wherein the cap is configured to snap onto the container.

What is claimed is:

1. A method for disinfecting a contact lens, the method comprising:
immersing the contact lens in a volume of from 2 mL to 20 mL of a disinfecting solution comprising from 1.5 vol % to 5 vol % H₂O₂ in a container in the presence of:
(i) a coated catalytic disc comprising:
a polymer support disc; and
a manganese oxide on at least a portion of the support disc, wherein the coated catalytic disc comprises from 6 mg to 300 mg of manganese; and
(ii) from 0.1 g to 0.5 g of a fibrous catalyst comprising from 18 to 50 mg of iron per gram of the fibrous catalyst.

2. The method of claim 1, wherein the coated catalytic disc contains from 60 mg to 250 mg of manganese.

3. The method of claim 1, wherein the manganese oxide comprises manganese (II) oxide (MnO), manganese (III) oxide (Mn₂O₃), manganese dioxide (MnO₂), or any combination thereof.

4. The method of claim 1, wherein the fibrous catalyst comprises polyacrylonitrile (PAN) fibers.

5. The method of claim 1, wherein an amount of the fibrous catalyst is from 0.1 g to 0.25 g.

6. The method of claim 1, wherein the fibrous catalyst comprises from 25 to 40 mg of iron per gram of the fibrous catalyst.

7. The method of claim 1, wherein the iron is covalently bound to the fibrous catalyst.

8. The method of claim 1, wherein the iron is an iron cation.

9. The method of claim 1, wherein a molar ratio of Mn:Fe is from 1:1 to 100:1.

10. The method of claim 1, wherein the immersing the contact lens is for a time sufficient to reduce H₂O₂ concentration to less than 50 ppm.

11. The method of claim 1, wherein the polymer support disc comprises a polyamide (PA), a polyphenylene oxide (PPO), a polyphenyl ether (PPE), a polypropylene (PP), a polyethylene (PE), a polystyrene (PS), or any combination thereof.

12. The method of claim 1, wherein the coated catalytic disc or the polymer support disc has a specific surface area in a range from 1 cm²/g to 20 cm²/g.

13. The method of claim 1, wherein the coated catalytic disc contains from 0.1 wt. % to 2 wt. % Mn.

14. The method of claim 1, wherein the coated catalytic disc is prepared by a process comprising:
(a) contacting the polymer support disc with a coating composition comprising a binder, a manganese oxide in flake form, and a diluent; and
(b) drying and/or curing to form the coated catalytic disc.

15. The method of claim 14, wherein the binder comprises an acrylic polymer and/or a urethane polymer.

16. The method of claim 14, wherein the diluent comprises water.

17. The method of claim 14, wherein the coating composition comprises from 0.05 wt. % to 5 wt. % of the manganese oxide.

18. The method of claim 14, wherein the coating composition further comprises an inert filler.

19. A contact lens disinfecting system comprising:
(a) a container configured to receive a disinfecting solution;
(b) a cap removably attachable to the container;
(c) a coated catalytic disc comprising:
a polymer support disc; and
a manganese oxide on at least a portion of the support disc, wherein the coated catalytic disc comprises from 6 mg to 300 mg of manganese; and
(d) from 0.1 g to 0.35 g of a fibrous catalyst comprising from 25 mg to 50 mg of iron per gram of the fibrous catalyst.

20. The system of claim 19, wherein:
the coated catalytic disc is removably attached to the cap; and
the fibrous catalyst is positioned at a bottom of the container.
